(19) United States
(12) Patent Application Publication
(10) Pub. No.: US 2020/0350044 A1
(43) Pub. Date: Nov. 5, 2020

(54) SYSTEM AND METHOD FOR HEALTH CARE DATA INTEGRATION AND MANAGEMENT

(71) Applicant: MEDecision, Inc., Wayne, PA (US)

(72) Inventors: David St. Clair, Narberth, PA (US); Kristel L. Schimmoller, Media, PA (US); Anita Nair-Hartman, Media, PA (US); Henry A. DePhillips, Wilmington, DE (US)

(73) Assignee: MEDecision, Inc., Wayne, PA (US)

(21) Appl. No.: 16/931,009

(22) Filed: Jul. 16, 2020

Related U.S. Application Data
(62) Division of application No. 15/616,700, filed on Jun. 7, 2017.
Application No. 15/616,700 is a continuation of application No. 11/493,922, filed on Jul. 27, 2006, now abandoned.
(60) Provisional application No. 60/702,640, filed on Jul. 27, 2005.

(51) Int. Cl. G16H 10/60; G16H 50/20; G16H 15/00

(52) U.S. Cl. CPC G16H 10/60; G16H 50/20; Y02A 90/26; Y02A 90/22; G16H 15/00

(57) ABSTRACT

In an exemplary embodiment, a method creates an electronic health record and analyzes that record to present a summary report. The report may optionally include treatment opportunities, strategies, and plans for the physician and patient. Data processing steps used to create and analyze the record and deliver the summary data may include aggregation, integration, internal validation, clinical validation, inspection, prediction, and communication.

This application claims the benefit of U.S. Provisional Patent Application Ser. No. 60/702,640 filed Jul. 27, 2005, titled “System and Method for Health Care Data Integration and Management,” which is incorporated by reference in its entirety.

### BACKGROUND OF THE INVENTION

### 1. Field of the Invention

The invention relates generally to the field of electronic health records, and in exemplary embodiments relates to improved methods for maintaining and delivering health record information.

### 2. Background Art

The Electronic Health Record (EHR) is a key element in efforts to manage health care delivery. In general, the EHR is most valuable today for the sickest members of our society—the 10% of the population that consumes 80% of the cost. With multiple conditions requiring multiple specialists, many powerful medications, numerous ancillary care providers and careful care coordination from case and disease managers, these individuals are also likely to be the least able personally to communicate the complexity of their histories and health status to their next treating physician. Yet it is exactly that complexity that confounds the medical community's attempts to reduce errors of omission and commission and to minimize the cost of duplicative and otherwise unnecessary care.

Broadly speaking, there are three sources of health care information about patients: the patients themselves (or their care givers); the patients' physicians, hospitals and other providers; and the patients' health plan or other payer.

Most patients have only limited information about their own care and even less ability to obtain, retain and store such data. Worse, a patient's ability to personally maintain their own health record decreases with illness, infirmity and, often, with age. Even the most user-friendly personal health record (PHR) systems available today are seldom used and even less frequently updated on a timely basis by their owners.

Physicians, hospitals and other providers are required by law and professional ethics to maintain significant records pertaining to the care they provide. These providers do not either generally or comprehensively obtain patient data from the spectrum of other providers. Thus, hospitals might have a deep reservoir of information regarding the services and tests provided to patients within the facility and, perhaps, by the admitting physician, but little, if any, information from other facilities or physicians who have treated those same patients. A single physician knows and has records of everything the patient has told him and the treatment he has provided, but that provider knows neither what the patient has been told by other physicians nor what treatment other physicians have provided. Complicating the distributed nature of the information is that it remains overwhelmingly paper-based and hand-written, rendering it exceedingly difficult to integrate, analyze and/or transmit effectively.

Therefore, there is a need for improved systems and methods for integrating patient data and providing it in a useful form to those who need it.

### SUMMARY OF THE INVENTION

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In an exemplary embodiment, a method creates an electronic health record (EHR), and analyzes that record to present treatment opportunities, strategies, and plans for the physician and patient in a Patient Clinical Summary report also referred to as a PCS report.

An electronic health record (EHR) may provide clinical information about an individual patient and may include data from various primary stakeholders in the healthcare system: Payers (insurance companies and other entities at financial risk for care), providers (physicians, pharmacists, nurses and other medical professionals in acute, ambulatory, nursing home, and home care settings), and the patients themselves.

Data residing at payers will be referred to as a payer-based health record (PBHR). The PBHR may include claims, care management, pharmacy, self-surveys and other data. Provider (hospitals and physician offices) data will be referred to as an electronic medical record (EMR). These records may include clinical findings, laboratory results, radiology images, or other data.

Finally, data that patients maintain on themselves are referred to as a personal health record (PHR). This data may include family medical history, patient medical history, over-the-counter medications, allergies, acute or chronic diseases, and other health and wellness information.

In an exemplary embodiment, a plurality of data processing steps are used to create the electronic health record and the summary. For example, these steps may include aggregation, integration, internal validation, inspection, prediction, and communication. In an embodiment, these patient-centric data processes correctly identify the patient and all the data from all sources that belong to that patient.

Aggregation is a process that collects data from one or more disparate sources that could belong to the patient in question. Preferably, aggregation takes all available data from all the components of the PBHR, EMR and PHR described above, and “weaves” them into an aggregate patient record that is utilized by subsequent data processes.

In an exemplary embodiment, an integration process examines the aggregate record for duplicate and overlapping data (e.g. identical laboratory results from both the lab and the doctor's office), identifies that data, eliminates the duplicate data, and assembles a revised single, consolidated record.

In an embodiment, internal validation processes are performed using various techniques, which may include probability assessment, referential edits, algorithms and/or other techniques to determine that certain key data elements in the consolidated record are, in fact, correct. For example, for some medical conditions such as heart attack, the “rule out” and “present” codes may be the same. Therefore, in exemplary embodiments, a mechanism to resolve ambiguity is desirable. In exemplary embodiments, an internal validation process is applied to drug data, laboratory results, physicians? assessments, and other data elements to conclude that a condition is or is not present. If, for example, there is one healthcare claim that indicates a condition, this may be insufficient information to conclude with certainty that the condition really exists. If, however, this claims data are corroborated by two or three physician encounter records or other healthcare data diagnosing the condition, then it is far more likely that the condition exists.

A similar validation process is used in exemplary embodiments to confirm that the patient in question is, indeed, the correct patient.

At the conclusion of these processes, in an exemplary embodiment, a single, integrated, woven, complete, and consolidated clinical history is provided for the correct patient. This record can then be used as a common, central electronic health record or EHR.

Inspection of the aggregated, integrated, and validated patient data in the consolidated record may then be accomplished as desired. In exemplary embodiments, this process compares the consolidated patient record to evidence-based guidelines and best practices to probe for gaps in care and reveal treatment opportunities. Some embodiments of the inspection process may identify care being delivered that is not appropriate as well as recommended care. In example embodiments, this process includes a hierarchy of prompts (alerts, warnings, and potential errors) that supports the physician's decision-making process.

In other exemplary embodiments, a prediction process may use various predictive modeling techniques (neural networks, artificial intelligence, etc.) to identify patients who are at higher risk than others for various conditions. In such cases, analytical techniques search for those patients who could require extensive medical services and identify the approximate cost of those services. The prediction process then identifies appropriate treatment strategies, plans and actions for the patient.

In exemplary embodiments, at the conclusion of the inspection and prediction processes, a summary (for example, a Patient Clinical Summary) of the analyzed consolidated patient record may be created for use by authorized individuals within the healthcare system.

In an embodiment, the summary is also forwarded to authorized individuals. Communication can be via the internet, smart card, fax, or in person with the display medium being a PC monitor screen, PDA device, or paper, for example.

The method disclosed is useful in creating a consolidated and validated electronic health record for a patient using data from one or more possibly inconsistent databases, and which may be summarized and communicated to any authorized health care provider, or the patient, as desired.

Additional features and advantages will be set forth in the description which follows, and in part will be apparent from the description, or may be learned by practice of the invention. These advantages will be realized and attained by the structure particularly pointed out in the written description and claims hereof as well as the appended drawings.

### BRIEF DESCRIPTION OF THE DRAWINGS

The accompanying drawings, which are incorporated herein and form a part of the specification, illustrate exemplary embodiments and, together with the description, further serve to explain principles that enable a person skilled in the pertinent art to make and use the invention.

FIG. 1 is a flow chart showing the operation of a disclosed process in an exemplary embodiment.

FIG. 2 is a block schematic diagram showing an apparatus for collecting, processing and distributing data.

FIG. 3 is a block schematic diagram showing a further embodiment of an apparatus for collecting, processing, and distributing data.

FIG. 4 is a block schematic diagram of a general purpose computer system used in some exemplary embodiments, and

FIG. 5 is a flow chart showing an exemplary embodiment of a process for electronically retrieving a patient clinical summary.

In the drawings, some like reference numbers indicate identical or functionally similar elements. Additionally, the left-most digit(s) of most reference numbers identify the drawing in which the reference numbers first appear.

### DETAILED DESCRIPTION OF THE PREFERRED EMBODIMENTS

The present invention will now be explained in terms of exemplary embodiments. This specification discloses one or more embodiments that incorporate the features of this invention. The embodiment(s) described, and references in the specification to “one embodiment”, “an embodiment”, “an example embodiment”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, persons skilled in the art may implement such feature, structure, or characteristic in connection with other embodiments whether or not explicitly described.

The application includes an appendix, which is part of this specification and is incorporated herein by reference.

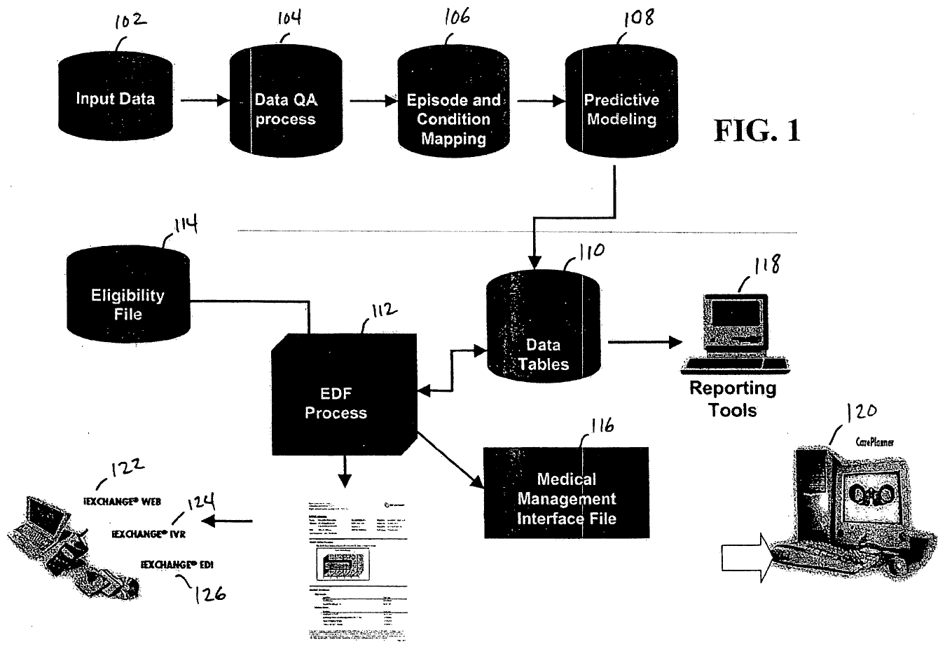

Referring to FIG. 1, in an exemplary embodiment, a method creates an electronic health record (EHR), and analyzes that record to provide desired outputs. For example, these outputs may include treatment opportunities, strategies, and plans for the physician and patient in a summary report.

The electronic health record (EHR) provides clinical information about an individual patient that may, for example, include data from three primary stakeholders in the healthcare system: Payers (insurance companies and other entities at financial risk for care), providers (physicians, pharmacists, nurses and other medical professionals in acute, ambulatory, nursing home, and home care settings), and the patients themselves.

Data residing at payers will be referred to as a payer-based health record (PBHR). The PBHR may include claims, care management, pharmacy, self-surveys and other data. Provider (hospitals and physician offices) data will be referred to as an electronic medical record (EMR). The EMR may include, for example, clinical findings, laboratory results, radiology images, and other data.

Finally, data that patients maintain themselves are referred to as a personal health record (PHR). This data may include, for example, family medical history, patient medical history, over-the-counter medications, allergies, acute or chronic diseases, and other health and wellness information.

In an exemplary embodiment, a plurality of data processing steps are used to create the electronic health record and the summary. For example, these steps may include one or more of aggregation, integration, internal validation, inspection, prediction, and communication. In exemplary embodiments these patient-centric data processes are combined to correctly identify the patient and all the data from all sources that belong to that patient.

In exemplary embodiments, aggregation is a process that collects all data from one or more disparate sources that could belong to the patient in question. In some preferred embodiments, aggregation takes all available data from all the components of the PBHR, EMR and PHR described above, and “weaves” them into an aggregate patient record that is utilized by subsequent data processes.

In an exemplary embodiment, an integration process examines the aggregate record for duplicate and overlapping data (e.g. identical laboratory results from both the lab and the doctor's office), identifies that data, eliminates the duplicate data, and assembles a revised single, consolidated record.

In an embodiment, internal validation processes are performed using various techniques, which may include probability assessment, referential edits, algorithms and/or other techniques to determine that certain key data elements in the consolidated record are, in fact, correct. For example, for some medical conditions such as a heart attack, the “rule out” and “present” codes used in some records may be the same. Therefore, it is desirable to provide a mechanism to resolve ambiguity. For example, internal validation may process drug data, laboratory results, physicians' assessments, and other data elements to conclude that the condition is or is not present. If, for example, there is one healthcare claim that indicates a condition, this may be insufficient information to conclude with certainty that the condition really exits. If, however, this claims data are corroborated by two or three physician encounter records or other healthcare data diagnosing the same condition, then it is much more likely that the condition exists.

A similar validation process is used in some embodiments to confirm that the patient in question is, indeed, the correct patient.

At the conclusion of these processes, in exemplary embodiments, a single, integrated, woven, complete, and consolidated clinical history exists on the correct patient. This record is referred to as the electronic health record or EHR.

Inspection of the aggregated, integrated, and validated patient data in the consolidated record may then be accomplished as desired. In exemplary embodiments, this process compares the consolidated patient record to evidence-based guidelines and best practices to probe for gaps in care and reveal treatment opportunities. The inspection process may identify care being delivered that is not appropriate as well as recommended care. This process may also include a hierarchy of prompts (alerts, warnings, and potential errors) that supports the physician's decision-making process.

In exemplary embodiments, a prediction process may use various predictive modeling techniques (neural networks, artificial intelligence, etc.) to identify patients who are at higher risk than others for various conditions. Analytical techniques search for those patients who could require extensive medical services and identify the approximate cost of those services. The prediction process then identifies appropriate treatment strategies, plans and actions for the patient.

At the conclusion of the inspection and prediction processes, a summary (for example, a Patient Clinical Summary) of the analyzed consolidated patient record is created for use by authorized individuals within the healthcare system.

In an embodiment, the summary is forwarded to authorized individuals. Communication can be via the internet, smart card, fax, or in person with the display medium being a PC monitor screen, PDA device, or paper, for example.

FIG. 1 is a data flow chart showing an exemplary embodiment of a generalized data flow method for processing health care data. This process may be implemented in a variety of embodiments that use one, several, or all of the steps shown in FIG. 1, and may include additional steps as desired, and otherwise be varied, all within the intended scope of the present invention.

As shown in the example of FIG. 1, the processing flow steps may include an input data step 102. In the example shown, a system obtains data from a variety of sources: copies of raw data from within the local system, copies of summary data in the local system, raw and summary data from data warehouses that payers or other providers control, and raw data transactions from production systems such as claims, pharmacy, lab results, EMRs, PHRs, and other data items.

Ideally, a unique and consistent patient identifier will be available for each record provided to the system. In many cases, the different systems involved may use disparate record formats and identifiers for those records. Thus, some level of processing and analysis is required to identify records belonging to the same individual and call for the relevant data from each. A Record Locator Service (RLS) may be used to indicate where there are records for “John Smith” and what identifiers are used to retrieve those records. As patient-authorized Unique Patient Identifier numbers become more widely implemented in the industry, this process can be simplified.

As shown in FIG. 1, the process then includes a data quality assurance step 104, episode and condition mapping step 106, and predictive modeling step 108. In an embodiment, the resulting data are put into tables in step 110. Data taken from an eligibility file in step 114 and the data tables from step 110 are used in an electronic records collection process in step 112 to produce a summary report and a medical management interface file in step 116. The summary report may be provided to recipients via web 122, interactive voice response (IVR) 124, electronic data interchange (EDI) 126, reporting tools 118, computer network connection 120, or other available methods.

Each data type has likely sources, and a framework is established in the system allowing gathering of data from a series of transactions of differing formats without destroying the integrity of the overall process. For instance, lab result data may come in from multiple sources (including various independent labs and hospital vendors), each with its own transaction characteristics. As standards are created for lab results data formats, the process is preferably adapted to integrate that data in the available format.

When a request for medical information is made by a provider, the system preferably retrieves all health records for a patient across a variety of sources. Four examples of data sources are: public health agencies, payers, providers, and the patients themselves. Public Health Agencies may include Medicare, Medicaid, NIH, CDC, etc. Payers may include managed care entities, PBMs, Labs, Referral and Authorizations, etc. Provider information may include Imaging Data, HIS, Referral and Authorization, Telemetry, and EMR. Patient data may include HRAs and Personal Health Records. Other sources may include grocery, financial transactions, etc.

The data are preferably assembled in a patient data model following a standard format. Many items of data will be associated with specific dates, often referred to as the Date of Service. Most lab tests, prescription fills, office visits, hospital stays and other health care services are performed at specific dates and times, and those dates can be very useful when assembling data for a patient. Some data (especially that from Personal Health Records) are not confined to specific dates. For example, family medical history, chronic illnesses, self-medication with Over The Counter (OTC) medications and dietary supplements may not be date-specific. Therefore, while time is a key dimension in the data assembly process, allowance must also be made for integrating data elements that do not have an associated date.

When the steps of getting data and assembling the data are complete, the data set is preferably processed further to eliminate redundancy and inconsistency. Lab results, for instance, may have been received both from the lab and from the doctor's office for the same test. The test results were originally created on the day after the doctor visit. For such cases, a set of rules are established to determine which record should be kept if there are multiple identical records, and which date should be used. Rules are also established for action in case minor differences are found between otherwise identical records.

The completion of these data integration functions preferably results in a single record, in a predetermined structure, that appears coherent from a data element perspective.

The data set is also tested as part of the integration process. The testing process preferably examines the data set to determine the clinical “truth” of the record. For example, the testing function may examine data to validate a level of certainty that the patient has a particular condition and to judge its severity. Elements are preferably cross checked within the EHR for this purpose. A finding that only one doctor has reported a chronic condition would indicate a higher level of uncertainty, while verification of the diagnosis by its presence on multiple claims or encounters across multiple physicians would increase certainty. Also, lab results that confirm the condition and prescription of appropriate drugs for some period of time each suggest a continuing belief that the condition exists.

An appropriate data testing process helps to eliminate the effects of coding errors, coding problems associated with “rule out” diagnoses, and misdiagnosis in the early stages of a condition (the example being a diagnosis of Schizophrenia for a patient later found to have Lyme Disease). It is desirable to have the capability to indicate, display and use (in analytics) a level of certainty regarding conditions, particularly if the system recommends treatment or further diagnosis based on these patient conditions.

In an embodiment, the result of the data testing step will be a patient's EHR in a standardized form that has been evaluated for consistency and has an identified likelihood of being correct. In some cases it is not possible to obtain complete records, but the data set available is preferably presented in a manner that is internally consistent and clinically reasonable.

Following the data testing step, the patient record (EHR) may be transmitted to its destination. The EHR may be transmitted to any authorized recipient. In particular, it may typically be available to at least three principal destinations: a subsequent evaluation step through the system (for identifying “treatment opportunities” by comparing it to Evidence-Based Medicine pathways and for predictive modeling), a third-party that requested the EHR, such as an emergency room or other care provider's office, and/or for saving to a data repository for reporting and/or later transmittal to one of the first two options. The entire available record, or any portion or summary of the record, may be transmitted as appropriate. In an exemplary embodiment, as described previously, a summary is provided to the care providers. This summary may have the format shown in Appendix A to this specification.

In an embodiment, the summary shares information the health plan has about the patient with the patient's treating clinicians. The summary is created and may apply evidence-based medicine and predictive modeling capabilities to identify treatment opportunities (“gaps in care”) for patients who are targeted for disease management or already enrolled in a disease management program. The treatment opportunities included within the Patient Clinical Summaries are intended to engage the physician in collaborating on the care plan for the patient. Various embodiments deliver the information in different forms depending on the needs of the recipient. In an embodiment, a web platform efficiently delivers health data to clinicians at the point of care.

This information may be delivered, for example, as a PDF report. Preferably, the presentation layer of the sending process supports HTML, PDF and XML output streams, as well as any other output streams that are desirable in view of the equipment and processes used by the recipients at the time. In an embodiment, the summary report is provided in an electronic data format compatible with a data processing system used by the receiving health care provider, such as in a raw data format so that the data can be immediately integrated into the provider's database.

The report (shown in Appendix A) has been formatted to meet the needs of clinicians by displaying the content of the report in an easy to use format that highlights the most critical information about the patient. The system may also provide this information electronically to other systems, such as patient portals and other provider information systems.

As shown in Appendix A, the summary may optionally include the following information:

Member Demographics: the most recent member demographic information.

Health Plan: the health plan from which this report was generated.

Health Status Measure: the HSM may be determined using the CaseAlert™ service available from MEDecision, the assignee of this application or other commercially available predictive modeling tool. The HSM is a score between 1-10 (10 is the highest) that shows the patient's risk relative to a master population.

Medical Conditions: displays medical conditions for which the patient has been treated. With each condition, a severity (Low, Moderate or High) is also displayed. The severity is based on the diagnosis code recorded on the healthcare claims, provider encounters or other healthcare data. For example diabetes with a diagnosis code of 250.00 is less severe than a diabetes diagnosis code of 250.10. The severity of the condition also takes into consideration any co-morbid conditions, the number of hospitalizations associated with the condition, the prescriptions and the lab values. In a preferred embodiment, various conditions are grouped according to their severity, so that high severity conditions are presented first in a group, followed by groups of lower severity conditions.

IP Facility Admissions: allows the provider to review any inpatient admissions that have occurred for the patient. This section also includes admit and discharge dates and the principal diagnosis.

Emergency Room Visits: provides information about emergency room visits by the patient.

Monitored Services: presents the provider with a list of services that which the patient has received. It also provides the last service date, the most recent servicing provider and that provider's phone number.

Medications: lists medications based on the USC code and description.

Providers Seen: lists providers recently seen by the patient. It includes provider specialty and phone number.

Clinical Flags: provides information regarding treatment opportunities and Preventative Health and Wellness clinical flags for the patient. Treatment opportunities identify clinical risk factors for the patient's condition and treatment guidelines, based on evidenced-based medicine. Preventative Health and Wellness clinical flags indicate generally healthy lifestyle services which the patient should receive. For example, a colonoscopy for a patient over the age of 50.

Care Management Summary: Based on the patient's identified condition(s) and associated severity, documents the care team's care plan for this patient.

Radiological and Laboratory Results: Lists results of radiological and laboratory tests.

With the goal to improve the efficiency of healthcare processes, reduce medical errors and decrease costs, providing immediate access to useful, timely, validated clinical information at the point of care will allow more appropriate clinical decisions resulting in improved clinical outcomes. Where return on investment has been difficult to demonstrate for EHR systems, the availability of easy-to-review summary data can have a significant impact on every clinician who will have access to a patient's history, medication list and other relevant clinical information. In fact, in a study produced by HealthCore on Jul. 24, 2006, the result of supplying summarized, validated payer EHR data in the emergency department (ED) setting was a reduction in the cost of the ED visit, together with the cost of the first day of hospitalization for the subset of patients who were admitted to the hospital, of five hundred and forty five dollars ($545) per Patient Clinical Summary provided.

The system supports privacy regulations and addresses privacy concerns by selectively limiting the display of information in the report. For example, conditions relating to mental or behavioral health or certain diseases such as AIDS may be filtered so that they are not displayed. Similarly, medications related to these conditions and providers may be omitted from the report where their inclusion would tend to disclose the occurrence of mental health or AIDS treatment.

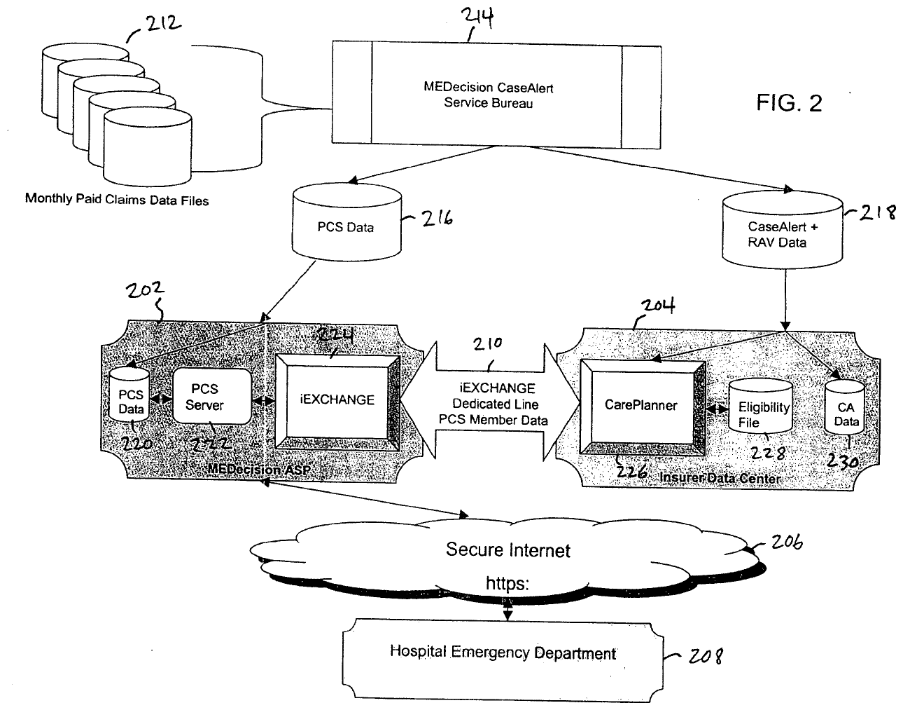

FIG. 2 is a block schematic diagram of one example implementation of a data processing and delivery system. In this embodiment, medical data processing is performed as a service using an Application Service Provider model. Various data files 212 are provided to a service bureau 214, which generates summary data, for example PCS data 216, in the manner described herein. The summary data set is provided to the ASP center 202. ASP center 202 comprises PCS data store 220, server 222 and internet delivery application 224. The data set is provided through delivery application 210 to one or more insurer data centers 204. In addition, further information can be obtained from those data centers for use in the summary. The summary is then provided to other parties as desired, for example via secure internet connection 206. The parties may include, as one example, a hospital emergency department 208 when a patient is brought in for treatment. A variety of parties may use the information provided and the applications are in no way limited to hospital emergency departments.

The insurer data center 204 receives case data from various sources. For example, the insurer data center 204 may receive claims data from the service bureau 214 through data store 218. The insurer data center 204 may comprise, among other things, interface 226, eligibility files 228, and case data store 230.

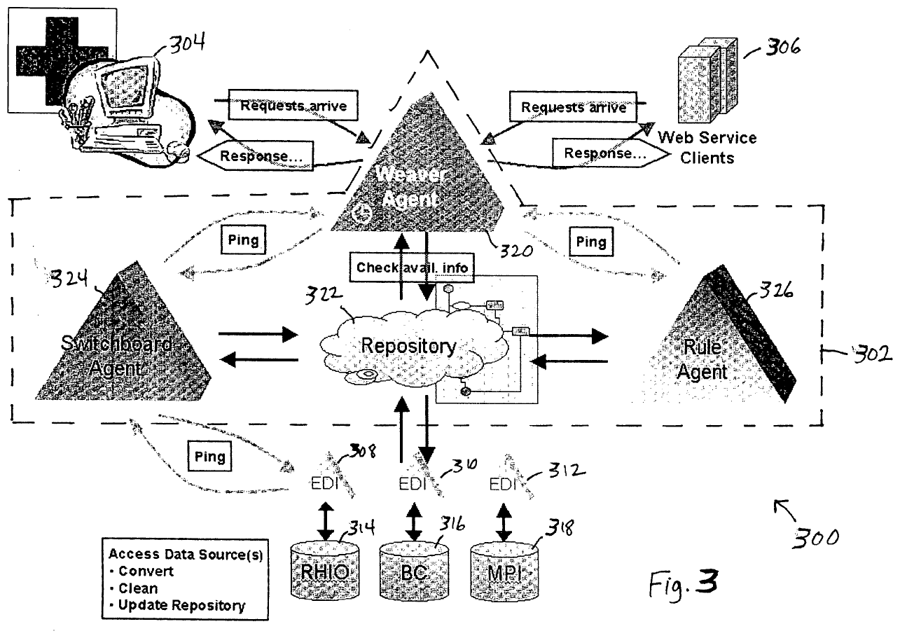

In one embodiment that may be more preferable than the embodiment of FIG. 2, a central processing center draws data on a real-time basis from a variety of sources and combines the data into useful records. These records are then made available to authorized persons. Referring to FIG. 3, central processing center 302 comprises weaver agent 320, switchboard agent 324, rule agent 326, and repository 322. These agent functions may be implemented in software, hardware, or a combination of the two. The agent functions may be combined in a single device or server, or may be divided as desired to be performed by one or more devices. Weaver agent 320 is connected to terminals 304 and to web clients 306 so that weaver agent 320 to receive requests from terminals 304 and web clients 306, process those requests, and provide responsive information.

Switchboard agent 324 is arranged to communicate with weaver agent 320, repository 322, and one or more electronic data interfaces (EDIs), for example EDI 308, EDI 310, and EDI 312. Rule agent 326 is arranged to communicate with weaver agent 320 and repository 322.

EDI 308 is an interface that obtains patient data from a regional health information organization or RHIO. EDI 312 is an interface to a patient identification mechanism and/or record locating service, for example a master patient index (MPI) 318. EDI 310 is an interface to a health insurer database, such as Blue Cross database 316.

In an embodiment, the functions of the switchboard agent include, but are not limited to, determining where to get information about a particular patient and obtaining the information when information about that patient is needed. Information about patients can be compiled in advance of a specific need, but to enhance privacy, preferably the information is gathered, processed, summarized and presented in real time only when needed.

The EDIs 308, 310 and 312 work with switchboard agent 324 to gather and assemble records from a variety of sources and translate them into a common format. The EDIs thus act as transfer agents, each providing a particular framework for interfacing with a disparate external database and retrieving desired information. The EDIs may include a programmable record parser, and may be configured to retrieve from an external record repository only those record fields or elements useful to the system. The EDIs may use data retrieved from an external source to populate one or more records defined by a class hierarchy or schema that has been established as a standardized record format for use in the system. The standardized record format will sometimes be referenced herein as an ontology. The EDIs may be provided with a table or other mapping mechanism that maps corresponding field names used in the external database to fields in the standard record ontology for the system. Of course, fields in the disparate systems may not match exactly. Fields may have different names, for example, one database may have a “compound” field while another uses the term “medications” for the same type of data. In an embodiment, the EDI provides a mapping between similar fields in the two databases. Fields from the external database may also be discarded, truncated, translated into a different format, or otherwise modified when they do not fit appropriately into the established ontology for the present system. Fields in the ontology for which no data are provided may be left empty. As a result, when an EDI communicates with an external data source to obtain information about a particular patient, it will provide to the repository 322 one or more records in a standard format following the established ontology.

In an embodiment, the standardized ontology includes the following categories of fields: Name, Patient Information, Date, Condition, Severity, Medication, Medication Class, Confidence, Care Plans, Radiological Studies, Laboratory Results, Biometrics and other Clinical Observations. Each of the field categories may have one or more data fields associated with it to hold desired information. For example, the Name category may be broken down into first name, middle name, and last name. Patient information may be broken down into date of birth, gender, a flag for multiple births, and a condition list. The condition category may include the condition name, start date, end date, severity level, and flags to show that the condition is “confirmed” and whether it is chronic. Medication may include date, medication name, and class of medication. Biometrics may include date taken, height, weight, and other biometric health monitoring or identifying information as desired.

An unlimited number of EDIs may be provided in the system, depending on the number of data sources that have agreed to participate and provide data. Standardized EDIs may be provided for data sources that follow a standard, such as the HL7 message standard. Customized EDIs may be provided for any external database that is arranged in a unique manner. As will be seen, the use of customized EDIs to translate disparate external database records into a standard format that can be readily processed by the weaver agent 320 makes it possible to more easily combine data from disparate sources to obtain a clear picture of the patient's status and history. The EDIs and other agents each define a core set of operations used between the agents and other frameworks to provide consistent yet flexible asynchronous operations. The use of a system built around autonomous agents also enhances the scalability of the system and its ability to operate using parallel processing.

If desired, the EDIs may provide data to the switchboard agent 324 and repository 322 using XML protocols, in accordance with the established ontology.

In an embodiment, rule agent 326 is an agent that implements clinical evidence-based care guidelines to provide, for example, treatment opportunities and recommendations, wellness and preventive recommendations, predictive health information, drug-to-drug interaction information, and other features. Such guidelines are commercially available or can be developed independently if desired, and incorporated into the rule agent 326.

Once the data has been collected in a standardized format in the manner just described, in an exemplary embodiment the group of records for a particular patient may be processed to eliminate any records not belonging that that patient, and to eliminate duplicate records. Then, the collected data may be assessed and validated. The validation process may include episode grouping, for example, if 20 services are provided during the same hospitalization incident they may be grouped for analytical purposes.

In some preferred embodiments, the validation process may include a clinical validation process to determine whether a record and an indicated diagnosis make sense in the context of other information available for the patient. The clinical validation process may also include condition confirmation analysis, to verify that a particular disease or condition is present before indicating that the condition is present in system outputs, such as for example a Patient Clinical Summary.

The inventors have identified particular criteria that are useful in establishing confirming condition logic to validate diagnoses contained in patient records, and other data suggesting that the patient may have a particular condition. As examples, the inventors have found the following information relevant. First, the frequency with which the diagnosis appears in the available data may be relevant. If two or three doctors have made the same diagnosis on the record, the diagnosis is more likely to be accurate than if it appears only once in the data. Lab test results, when available, may also be used to confirm some diagnoses. Pharmacy claims and pharmacy sales and dispensing records may also be used for confirmation of a diagnosis, as particular medications and items provided by pharmacies are specific to, or suggestive of, particular conditions. Radiology results may also be used to confirm a condition that can be identified through radiology, such as various forms of cancer, strokes, etc. Submission of data by specialty providers is also an indicator that can be used to confirm a patient condition. For example, if an encounter from an oncology specialist is present in the record, it is more likely that data suggesting a cancer condition is correct. Finally, hospital discharge diagnoses can be used to confirm conditions. As additional sources of data and additional categories of data are added to the system, further confirming criteria can be added to the foregoing. The criteria to be used for validating data indicating a specific condition may be selected from among the foregoing criteria, or other criteria may be used, within the scope of the invention.

In an exemplary embodiment, the criteria to be used are selected from among the foregoing confirming data types. These criteria are then placed in a hierarchy most appropriate for the possible diagnosis under evaluation.

Table 1 shows an exemplary embodiment of a hierarchy of these criteria for validating diagnoses of the ten most common medical conditions.

TABLE 1

| | | | | | Pharmacy | | | |
|---|---|---|---|---|---|---|---|---|
| | | | Freq. of | Lab Test/ | Claim or | Radiology | Submission | Hospital |
| | | ICD 9 | Diagnosis | Result | Prescription | Results | by Specialty | Discharge |
| | Top 10 Conditions/Diagnosis | Code | in Data | Confirm | Confirm. | Confirm. | Providers | Diagnosis |
| 1 | Coronary Artery Disease (CAD) | 413.9-414.0 | 3 | 5 | 4 | 6 | 1 | 2 |
| 2 | Heart Failure | 428.0-428.9; | 4 | 6 | 5 | 3 | 1 | 2 |
| | | 402.11 | | | | | | |
| 3 | Lung Cancer | 162-162.9 | 4 | 6 | 5 | 2 | 1 | 3 |
| 4 | Breast Cancer | 174-175 | 4 | 5 | 6 | 2 | 1 | 3 |
| 5 | Cerebral Vascular Disease-Stroke | 429.2; | 4 | 6 | 5 | 3 | 1 | 2 |
| | | 430-438 | | | | | | |
| 6 | COPD | 491.21, 496 | 4 | 5 | 6 | 3 | 1 | 2 |
| 7 | Diabetes | 250-250.9 | 4 | 2 | 5 | 6 | 1 | 3 |
| 8 | Pneumonia | 480.0-480.9, 481, | 4 | 6 | 5 | 2 | 1 | 3 |
| | | 482.0-482.9, | | | | | | |
| | | 483.0-483.8, 485 | | | | | | |
| 9 | Alzheimer's Disease | 331 | 4 | 6 | 3 | 5 | 1 | 2 |
| 10 | Renal Failure | 584-586, 593.9 | 4 | 2 | 6 | 5 | 1 | 3 |

As can be seen upon inspection of Table 1, the criteria may be weighted differently when evaluating possible diagnoses for different diseases. For example, persons diagnosed with lung cancer typically have radiology tests showing the disease, so that radiology information would be strong evidence supporting that diagnosis, while a lack of radiology information in the patient file would raise suspicions about whether the patient has really been diagnosed with lung cancer. In contrast, radiology information in the patient's record is unlikely to either confirm or deny a diagnosis of diabetes or coronary artery disease.

The hierarchy of confirmation established in Table 1 (confirming diagnosis) is based on the confidence level of a definitive diagnosis utilizing a scoring/weighting range of 1-6. A score of 1 indicates the highest confidence level for a positive diagnosis with a score of 6 having the least reliable value for validating or verifying the diagnosis. The system may generate a weighted confidence level parameter that a condition exists, based on the evidence available and the weight it has been assigned.

Table 2 illustrates exemplary clinical validation rules for diabetes that can be used, for example, in conjunction with the weighting established in the hierarchy of Table 1 to determine a confidence level that a condition is present.

In order for a condition to be deemed to have a high confidence level for being present, certain combinations of weighted factors must exist. For example, in diabetes, if weighted factors 1-3 (Specialty Provider diagnosis, lab test or result confirmation, hospital discharge diagnosis) are present in combination with any other factor in table 1 for diabetes, the confidence level threshold is considered to be surpassed and the diagnosis is considered to be validated or confirmed.

If the confidence level based on the available records exceeds a predetermined threshold level, the condition is considered “confirmed” and a flag associated with that condition in the patient's record is set to indicate that the condition can be included on summary documents with a reasonable level of confidence that the patient has been diagnosed with that condition. The confidence level may be determined using the weighted method describe above, or based on a certain number of validating factors. For example, a diagnosis of diabetes could be confirmed if any three of the factors identified in Table 2 are found in the patient's records.

TABLE 2

| | Frequency of | | Pharmacy Claim | Radiology | Submission | Hospital |
|---|---|---|---|---|---|---|
| | Diagnosis in | Lab Test/Result | of Prescription | Results | by Specialty | Discharge |
| | Data | Confirmation | Confirmation | Confirmation | Providers | Diagnosis |
| 1 | Occurrence of | FBS > 300 | Combination of | None | ICD 250.0-250.1 | Principal D/C |
| | (3) or more | (CPT 82947) | Insulin + Syringe + Pen | | by Specialist in | Diagnosis |
| | Diabetes | | needle (See below) | | Endocrinology | with ICD |
| | diagnoses | | | | | code 250.0- |
| | | | | | | 250.1 |
| 2 | | A1C >= 9% | Any Hypoglycemic | | | |
| | | (CPT code | agent | | | |
| | | 83036-83037) | | | | |
| 3 | | Fructosamine >= 500 | Test Strips (for | | | |
| | | (CPT 82985) | blood glucose | | | |
| | | | monitors) | | | |

In this manner, the system can validate diagnoses and other condition information suggested by one or more records for the patient and substantially prevent erroneous statements of patient condition from appearing on summary documents.

Specific items dispensed at pharmacies may be identified that are associated with each disease. The recorded delivery of these items may be evidence that a diagnosis suggested by other data should be confirmed. For example, for diabetes, various hypoglycemic agents that are typically prescribed may be identified and listed in a table. The table may also include, for example, various pen type needles that are commonly used by patients with diabetes. For example, the following items may be included in the table for diabetes: Pen Needles, Bd Original Pen Needles, Bd Short Pen Needles #08290-3201-09, Bd Short Pen Needles 5 mm, Exel Insul Pen Needles 8 mm, Kroger Pen Needles 29 g, Kroger Pen Needles 31 g, Leader Pen Needles 29 g, Leader Pen Needles 31 g, Pen Needles 12 mm 29 g, Pen Needles 6 mm 31 g, and Pen Needles 8 mm 31 g.

Similarly, various brands and types of insulin may be included in the table to assist in confirming the diagnosis. For example, the following pharmacy dispensing codes are among those used for insulin: 54868-1428-*1, 12854-*335-00, 12854-*335-01, 12854-*335-34, 14608-335, 00088-2220-01, 00088-2220-34, 00088-2220-33, 00002-8310-01, 00420-3687-12, 00420-3687-92, 00169-3687-12, and 00169-3687-92.

As another example, the dispensation and use of test strips may provide information useful in confirming or ruling out a diagnosis of diabetes. The following are examples of test strips that are frequently used by patients with diabetes: Accu-Chek Advantage Strips, Accu-Chek Aviva Test Strips, Accu-Chek Compact Strips, Advance Micro-Draw Test Strips, Advance Test Strips, Albustix Reagent Strips, Ascensia Autodisc Strips, Ascensia Contour Strips, Ascensia Elite Test Strips, Assure 3 Test Strips, At-Last Test Strips, Bd Test Strips #53885-0245-10, Control Test Strips, Cvs Blood Glucose Strips, Diastix Reagent Strips, Easygluco Test Strips, Easypro Test Strips, Fast Take Test Strips, Freestyle Test Strips, Glucostix Reagent Strips, Keto-Diastix Reagent Strips, Ketocare Ketone Test Strips, Kinray Test Strips, Kroger Test Strips, Labstix Reagent Strips, Multistix 10 Sg Reag Strips, Multistix Reagent Strips, Nexgen Glucose Test Strips, One Touch Test Strips, One Touch Ultra Test Strips, Precision Pcx Test Strips, Precision Q-I-D Test Strips, Precision Xtra Test Strips, Prestige Test Strips, Reli On Test Strips, Shoprite Test Strips, Surestep Test Strips, Truetrack Glucose Strips, Truetrack Test Strips, Ultima Test Strips, Uristix 4 Reagent Strips, and Uristix Reagent Strips.

Embodiments of the disclosed system may be implemented in hardware, firmware, software, or any combination thereof. Embodiments of the invention may also be implemented as instructions stored on a machine-readable medium, which may be read and executed by one or more processors. A machine-readable medium may include any mechanism for storing or transmitting information in a form readable by a machine (e.g. a computing device). For example, a machine-readable medium may include read only memory (ROM); random access memory (RAM); hardware memory in PDAs, mobile telephones, and other portable devices; magnetic disk storage media; optical storage media; flash memory devices; electrical, optical, acoustical, or other forms of propagated signals (e.g. carrier waves, infrared signals, digital signals, analog signals, etc.), and others. Further, firmware, software, routines, instructions, may be described herein as performing certain actions. However, it should be appreciated that such descriptions are merely for convenience and that such actions in fact result from computing devices, processors, controllers or other devices executing the firmware, software, routines, instructions, etc.

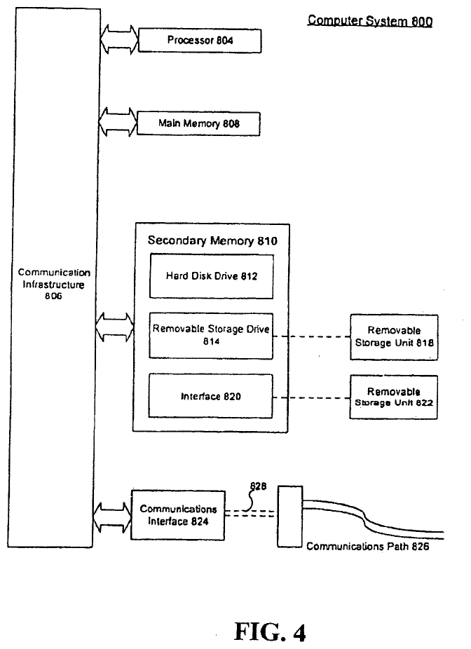

The following description of a general purpose computer system is provided for completeness. The disclosed systems and methods can be implemented as software, in hardware, or as a combination of software and hardware. Consequently, the disclosed system may be implemented in the environment of a computer system or other processing system. In one exemplary embodiment, the computers and devices shown in FIGS. 1-3 may be personal computers, servers or other computing system. An example of such a computer system is shown at reference number 800 in FIG. 4. In the disclosed systems, all of the elements depicted in FIGS. 1-3, for example, can execute on one or more distinct computer systems 800, to implement the various disclosed methods. The computer system 800 includes one or more processors, such as a processor 804. The processor 804 can be a special purpose or a general purpose digital signal processor. The processor 804 is connected to a communication infrastructure 806 (for example, a bus or network). Various software implementations are described in terms of this exemplary computer system. After reading this description, it will become apparent to a person skilled in the relevant art how to implement the disclosed systems and methods using other computer systems and/or computer architectures.

The computer system 800 also includes a main memory 808, preferably random access memory (RAM), and may also include a secondary memory 810. The secondary memory 810 may include, for example, a hard disk drive 812 and/or a removable storage drive 814, representing a floppy disk drive, a magnetic tape drive, an optical disk drive, etc. The removable storage drive 814 reads from and/or writes to a removable storage unit 818 in a well known manner. The removable storage unit 818, represents a floppy disk, magnetic tape, optical disk, etc. which is read by and written to by the removable storage drive 814. As will be appreciated, the removable storage unit 818 includes a computer usable storage medium having stored therein computer software and/or data.

In alternative implementations, the secondary memory 810 may include other similar means for allowing computer programs or other instructions to be loaded into the computer system 800. Such means may include, for example, a removable storage unit 822 and an interface 820. Examples of such means may include a program cartridge and cartridge interface (such as that found in video game devices), a removable memory chip (such as an EPROM, or PROM) and associated socket, and other removable storage units 822 and interfaces 820 which allow software and data to be transferred from the removable storage unit 822 to the computer system 800.

Computer system 800 may also include a communications interface 824. Communications interface 824 allows software and data to be transferred between the computer system 800 and external devices. Examples of communications interface 824 may include a modem, a network interface (such as an Ethernet card), a communications port, a PCMCIA slot and card, or other communications path interface devices. Software and data transferred via the communications interface 824 are in the form of signals 828 which may be electronic, electromagnetic, optical or other signals capable of being received by communications interface 824. These signals 828 are provided to communications interface 824 via a communications path 826. Communications path 826 carries signals 828 and may be implemented using wire or cable, fiber optics, a phone line, a cellular phone link, an RF link and other communications channels.

In this document, the terms computer program medium and computer readable medium are used to generally refer to media such as the removable storage drive 814, a hard disk installed in hard disk drive 812, and the signals 828. These computer program products are means for providing software to the computer system 800.

Computer programs (also called computer control logic) are stored in the main memory 808 and/or the secondary memory 810. Computer programs may also be received via the communications interface 824. Such computer programs, when executed, enable the computer system 800 to implement the disclosed systems and methods as discussed herein. In particular, the computer programs, when executed, enable the processor 804 to implement the processes disclosed herein. Accordingly, such computer programs operate to control computer system 800. By way of example, in various exemplary embodiments, the processes/methods performed by signal processing blocks of encoders and/or decoders can be performed by computer control logic. Where the disclosed systems and methods are implemented using software, the software may be stored in a computer program product and loaded into the computer system 800 using the removable storage drive 814, the hard drive 812 communications interface 824, or any other known method of transferring digital information into a computer system.

In another embodiment, disclosed features are implemented primarily in hardware using, for example, hardware components such as Application Specific Integrated Circuits (ASICs) and gate arrays. Implementation of a hardware state machine so as to perform the functions described herein will also be apparent to persons skilled in the relevant art(s).

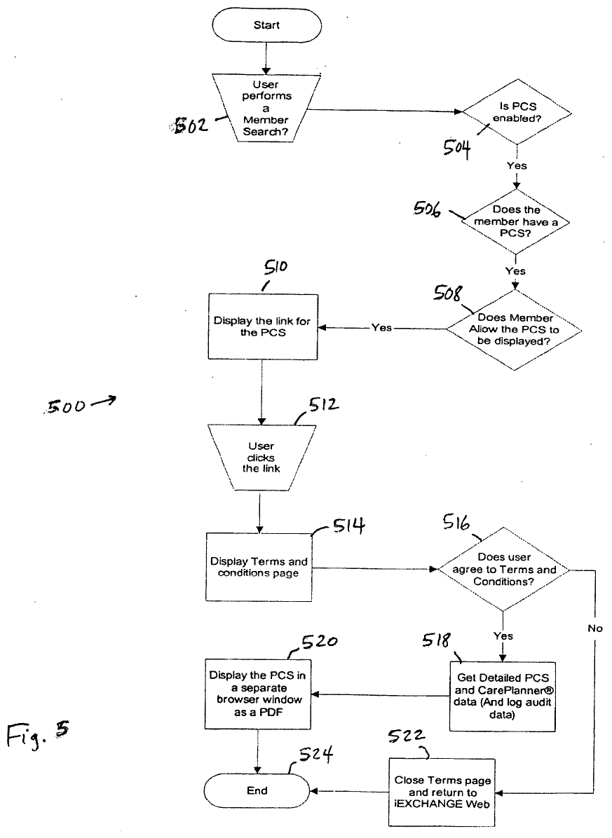

FIG. 5 shows a flow chart for a process of retrieving and displaying Patient Clinical Summary (PCS) information. In step 502, an authorized user of the system performs a search for a patient or “member.” In step 504, the system determines whether the PCS is available to the user for that patient. If so, at step 506, the system determines whether the system has an existing PCS for the member or can generate one. If one is available, the process continues at step 508 and the system determines whether the member has authorized use of the PCS. If so, the process continues at step 510 and a link to the PCS is displayed. If, in steps 504, 506, or 508, the PCS is not available, the process ends at step 524.

In response to the display of the link at step 510, the user clicks on the link at step 512. A terms and conditions page is displayed at step 514, and in step 516, the system requests acceptance of the terms and conditions. If they are accepted, the process continues at step 518 and detailed data are retrieved. The PCS is displayed in step 520, for example in a separate window, and the process ends at step 524. If the terms are not accepted in step 516, the process continues at step 522, the link is closed, and the process ends with step 524.

While various embodiments have been described above, it should be understood that they have been presented by way of example only, and not limitation. Thus, the breadth and scope of the present invention should not be limited by any of the above-described exemplary embodiments, but should be defined only in accordance with the following claims and their equivalents.

1-29. (canceled)

30. A method for operating a medical record system, comprising:
collecting a plurality of medical history data records for a single patient from a plurality of sources;
selecting from at least one of the data records a diagnosis code representing a possible medical condition of the patient;
mapping a data field from one of the plurality of medical history data records to a plurality of non-identical data fields for each of the other medical history data records as a function of a predetermined correlation between the data field and the plurality of non-identical data fields, each of the data field and the plurality of non-identical data fields containing data associated with a predetermined medical condition, and storing the data from the data field and each of the non-identical data fields from each of the plurality of medical history data records to a patient care summary record;
electronically analyzing the patient care summary record to identify confirming information supporting an analysis that the patient has the medical condition represented by the diagnosis code and contradicting information supporting an analysis that the patient does not have the medical condition represented by the diagnosis code; and
generating a confidence score that the patient can be presumed to have the medical condition represented by the diagnosis code as a function of the confirming information and the contradicting information.

31. The method of claim 30, wherein the confirming information includes information representing a frequency of appearance of the diagnosis code in the data records.

32. The method of claim 31, wherein a plurality of said categories of confirming information are selected based on the diagnosis code under evaluation.

33. The method of claim 32, wherein information in one of said categories of confirming information is weighted differently from information in another category of confirming information depending on the diagnosis code under evaluation.

34. The method of claim 33, wherein a hierarchy of relative value in determining whether the patient has the medical condition represented by the condition code is assigned to said categories of confirming information.

35. The method of claim 31, wherein available confirming information is evaluated based on predetermined clinical validation rules.

36. The method of claim 35, wherein available contradicting information is evaluated based on predetermined clinical validation rules.

37. The method of claim 30, wherein said output includes a summary report identifying medical conditions that the patient can be presumed to have based on analysis of confirming information in the data records for that patient.

38. The method of claim 30 wherein selecting from at least one of the data records the diagnosis code representing the possible medical condition of the patient further comprises determining whether the diagnosis code is present in at least two of the data records.

39. The method of claim 38 further comprising generating the confidence score as a function of the number of data records that contain the diagnosis code.

40. The method of claim 30, wherein the confirming information includes information representing a predetermined lab test result.

41. The method of claim 30, wherein the confirming information includes information representing predetermined pharmacy claims or prescriptions data.

42. The method of claim 30, wherein the confirming information includes information representing predetermined radiology results.

43. The method of claim 30, wherein the confirming information includes information representing a submission by a specialty provider.

44. The method of claim 30, wherein the confirming information includes information representing hospital discharge diagnosis data.

45. The method of claim 30 wherein generating the confidence score comprises placing a greater weight on the confirming information than on the contradicting information.

45. The method of claim 30 wherein generating the confidence score comprises placing a greater weight on a submission by a specialty provider than on other confirming information.

46. The method of claim 30 wherein generating the confidence score comprises placing a greater weight on a submission by a specialty provider and a hospital discharge diagnosis than on other confirming information.

47. The method of claim 30 wherein generating the confidence score comprises placing a greater weight on a submission by a specialty provider and a radiology result than on other confirming information.

48. The method of claim 30 wherein generating the confidence score comprises placing a greater weight on a submission by a specialty provider and a lab test than on other confirming information.
